(19) United States
(12) Patent Application Publication
(10) Pub. No.: US 2024/0309334 A1
(43) Pub. Date: Sep. 19, 2024

(54) METHOD OF REPROGRAMMING CELLS

(71) Applicant: ReGen Theranostics, Inc., Rochester, MN (US)

(72) Inventor: Timothy Nelson, Rochester, MN (US)

(73) Assignee: ReGen Theranostics, Inc., Rochester, MN (US)

(21) Appl. No.: 18/121,340

(22) Filed: Mar. 14, 2023

(51) Int. Cl. C12N 5/074; A01N 1/02; C12N 15/86

(52) U.S. Cl. CPC C12N 5/0696; A01N 1/0284; C12N 15/86; C12N 2501/115; C12N 2501/15; C12N 2501/60; C12N 2506/1307; C12N 2740/15043

(57) ABSTRACT

In some embodiments, the present disclosure is directed to methods of reprogramming fibroblasts into induced pluripotent stem cells. In some embodiments, a method comprises obtaining fibroblasts, such as from a skin biopsy. In some embodiments, a method comprises reprogramming fibroblasts using a viral vector encoding reprogramming factors. In some embodiments, a method comprises expanding iPSC and identifying confluent iPSC.

### FIELD OF THE INVENTION

The present invention generally relates to the field of regenerative medicine. In particular, the present invention is directed to a method of generating induced pluripotent stem cells.

### BACKGROUND

Induced pluripotent stem cells have a variety of uses in regenerative medicine.

### SUMMARY OF THE DISCLOSURE

In an aspect, disclosed herein is a method of reprogramming cells, the method comprising: obtaining a plurality of fibroblast cells; reprogramming the plurality of fibroblast cells by delivering to the plurality of fibroblast cells a polynucleotide encoding a reprogramming factor, and transferring the plurality of fibroblast cells to a reprogramming medium, thereby generating a plurality of iPSC; expanding the plurality of iPSC; and identifying a plurality of confluent iPSC from the plurality of iPSC.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a diagram illustrating an exemplary method of reprogramming cells;

FIGS. 2A-2B are diagrams illustrating exemplary methods of reprogramming cells;

FIG. 3 is a diagram illustrating an exemplary method of collecting, expanding, and freezing fibroblast cells;

FIG. 4 is a diagram illustrating an exemplary method of reprogramming cells from a starting point of frozen fibroblast cells;

FIG. 5 is a diagram illustrating viral delivery of a polynucleotide to a cell;

FIG. 6 is a diagram illustrating an exemplary method of reprogramming cells;

FIG. 7 is a diagram illustrating an exemplary system for automating a method of reprogramming cells; and

FIG. 8 is a diagram depicting elements of an exemplary computer system.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to a method of reprogramming cells.

In some embodiments, aspects of the present disclosure are directed to a method of generating induced pluripotent stem cells. As used herein, the term “induced pluripotent stem cell” (iPSC) has a meaning well-known in the art and refers to cells having properties similar to those of embryonic stem cells (ESC) and encompasses undifferentiated cells artificially derived by reprogramming differentiated, non-pluripotent cells. As used herein, a “differentiated” cell is a cell that takes on a more committed (“differentiated”) position within a given cell lineage.

In some embodiments, a method described herein reprograms fibroblasts into iPSC. As used herein, the term “reprogramming” means a process that alters or reverses the differentiation status of a somatic cell that is either partially or terminally differentiated. Reprogramming of a somatic cell may be a partial or complete reversion of the differentiation status of the somatic cell. In some embodiments, reprogramming is complete when a somatic cell is reprogrammed into an induced pluripotent stem cell. However, reprogramming may be partial, such as reversion into any less differentiated state. For example, reverting a terminally differentiated cell into a cell of a less differentiated state, such as a multipotent cell. As used herein, the term “somatic cell” refers to any cell other than pluripotent stem cells or germ cells. In some embodiments, a somatic cell is a fibroblast.

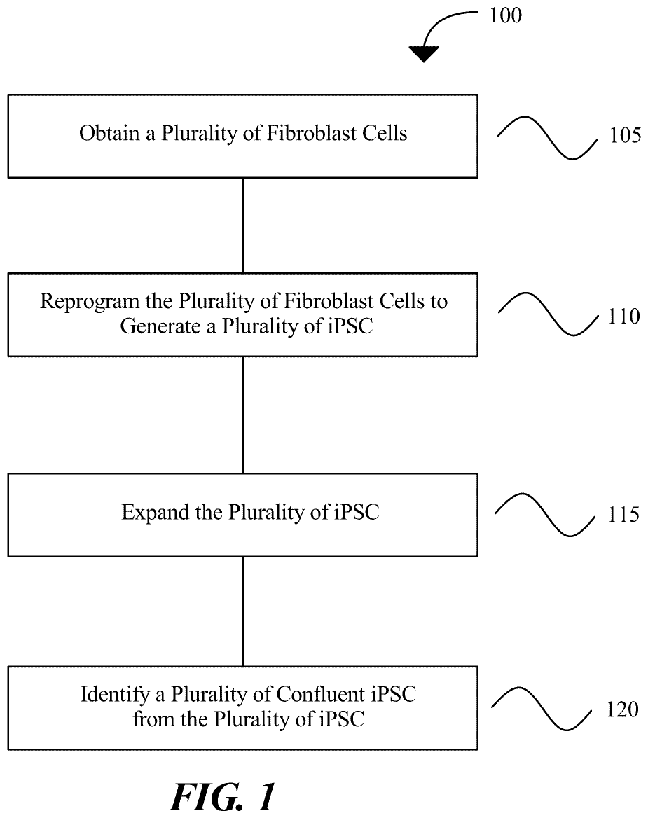

Referring to FIG. 1, fibroblasts may be reprogrammed into iPSC via a method described herein. Fibroblasts are cells that contribute to the formation of connective tissue. Among other functions, fibroblasts aid in wound healing and secrete a number of proteins including collagen. Fibroblasts may be reprogrammed into iPSC by a method 100 including the following steps: obtaining a plurality of fibroblast cells 105, reprogramming the plurality of fibroblast cells by delivering to the plurality of fibroblast cells a polynucleotide encoding a reprogramming factor, and transferring the plurality of fibroblast cells to a reprogramming medium, thereby generating a plurality of iPSC 110, expanding the plurality of iPSC 115, and identifying a plurality of confluent iPSC from the plurality of iPSC 120. In some embodiments, a method may be carried out in this order. In some embodiments, a method may further include a step of freezing a plurality of confluent iPSC. In some embodiments, a method may further include a step of differentiating a plurality of confluent iPSC. In some embodiments, a method further includes a step of differentiating confluent iPSC into cardiac lineage cells. In some embodiments, a polynucleotide encoding a reprogramming factor is RNA (such as mRNA) encoding a reprogramming factor.

Still referring to FIG. 1, a plurality of fibroblasts may be obtained using any method that may occur to persons skilled in the art upon reviewing the entirety of this disclosure. For example, and without limitation, a plurality of fibroblasts may be obtained via a skin biopsy.

Still referring to FIG. 1, a plurality of fibroblasts, or a sample containing a plurality of fibroblasts, may undergo an initial processing step. A sample containing a plurality of fibroblasts, such as a biopsy, may be cut. A sample containing a plurality of fibroblasts may undergo a first expansion step, such as by being placed into one or more wells containing a first growth medium. A first growth medium may include one or more of CTS knockout DMEM, CTS Glutamax, and PLTGold. A first expansion step may allow fibroblasts in a sample to multiply. A first expansion step may include one or more medium changes. A first expansion step may include medium changes on days 6, 12, and 18 post initial processing step. An first expansion step may include transfer of a plurality of fibroblasts into a vessel or division of a plurality of fibroblasts into multiple vessels. For example, a plurality of fibroblasts may be transferred into a T25 flask on day 9 post initial processing step. For example, a plurality of fibroblasts may be divided into 4 T25 flasks on day 15 post initial processing step.

Still referring to FIG. 1, a plurality of fibroblasts may be tested. For example, a plurality of fibroblasts may undergo one or more of sterility, viability, cell count, mycoplasma, karyotype, and DNA fingerprinting testing.

Still referring to FIG. 1, a plurality of fibroblasts may be frozen. For example, a plurality of fibroblasts may be frozen on day 21 post initial processing step. A plurality of fibroblasts may be frozen in a freezing medium, such as Cryotor CS10.

Still referring to FIG. 1, a plurality of fibroblasts may be reprogrammed into a plurality of iPSC.

Still referring to FIG. 1, a plurality of fibroblasts may be thawed. For example, a plurality of fibroblasts may be thawed on day 6 before infection.

Still referring to FIG. 1, a plurality of fibroblasts may undergo a second expansion step, such as by being placed into one or more wells containing a second growth medium. A second growth medium may include one or more of CTS knockout DMEM, CTS Glutamax, and PLTGold. A second expansion step may allow fibroblasts and/or iPSC in a sample to multiply.

Still referring to FIG. 1, a plurality of fibroblasts may be infected with one or more viral vectors encoding one or more reprogramming factors. For example, a plurality of fibroblasts may be infected with one or more viral vectors encoding one or more reprogramming factors during a second expansion step.

Still referring to FIG. 1, a viral vector may include a lentiviral vector. A viral vector may include a sendaiviral vector. A viral vector may include a rendoviral vector. A viral vector may be from an RNA virus. RNA viruses do not require integration of viral DNA into a host genome. In some embodiments, use of an RNA virus (rather than a DNA virus) may prevent the possibility of integration into an important host gene and allow viral genetic material to be more easily cleared.

Still referring to FIG. 1, a viral vector may encode one or more reprogramming factors. For example, a viral vector may encode one or more of hOct3/4, hSox2, hKlf4, and hc-Myc. As used herein, the term “reprogramming factor” refers to a molecule, which when contacted with a cell, or produced in a cell from exogenous DNA or RNA (e.g., produced from transduced RNA), can, either alone or in combination with other molecules, cause reprogramming. A viral vector may encode a reprogramming factor selected from the list consisting of Oct3 protein, Oct4 protein, Myo-D-Oct4 protein, Sox1 protein, Sox2 protein, Sox3 protein, Sox15 protein, Klf1, protein, Klf2 protein, Klf3 protein, Klf4 protein, Klf5 protein, c-Myc protein, L-Myc protein, N-Myc protein, Nanog protein, Lin28A protein, Tert protein, Utf1 protein, Aicda protein, Glis1, Sall4, Esrrb, Tet1, Tet2, Zfp42, Prdm 14, Nr5a2, Gata6, Sox7, Pax1, Gata4, Gata3, cEBPa, HNF4a, GMNN, SNAIL, Grb2, Trim71, and biologically active fragments, analogues, variants, and family members thereof. Exposure of a plurality of fibroblasts to a viral vector encoding one or more reprogramming factors may result in reprogramming of fibroblasts into iPSC.

Still referring to FIG. 1, a second expansion step may include one or more medium changes. For example, a medium may be changed on day 4 before infection. For example, media may be changed on days 1, 2, 4, and 6 post infection. A plurality of fibroblasts or a plurality of iPSC may be replated during a second expansion step. For example, a plurality of fibroblasts may be replated on day 2 before infection. A plurality of fibroblasts or a plurality of iPSC may be transferred to a plate containing an extracellular protein matrix (such as laminin, collagen, fibronectin, etc.). For example, a plurality of fibroblasts or a plurality of iPSC may be transferred to a plate containing a laminin 521 matrix. A plurality of fibroblasts or a plurality of iPSC may be transferred to a plate containing laminin 521 on day 7 post infection. Media changes and cell division may aid in clearing or diluting a portion of a viral vector. In some embodiments, media changes may be done until the viral vector has been cleared. A plurality of fibroblasts or a plurality of iPSC may adhere to an intracellular protein matrix such that the plurality of fibroblasts or the plurality of iPSC is retained on a plate through a medium change.

Still referring to FIG. 1, a medium in a vessel containing a plurality of fibroblasts or a plurality of iPSC may be changed to a reprogramming medium. As used herein, a reprogramming medium is a medium that is conducive to iPSC expansion. In some embodiments, exposure of fibroblasts to reprogramming factors and culture in reprogramming media causes fibroblasts to be reprogrammed into iPSC and expand. A reprogramming medium may include one or more of L-ascorbic acid-2-phosphate magnesium, sodium selenium, FGF2, insulin, NaHCO₃, transferrin, TGFβ1, and NODAL. A reprogramming medium may be based on a DMEM/F12 medium. Exemplary reprogramming media include mTeSR1, STEMPRO, Tesr E7 and Tesr E8. A reprogramming medium may be configured to cause iPSC reprogrammed from fibroblasts via reprogramming factors to expand. In some embodiments, cell expansion includes cell growth and multiplication, and may be caused by, for example, culturing cells in a medium containing nutrients and a matrix. A medium in a vessel containing a plurality of fibroblasts or a plurality of iPSC may be changed to a reprogramming medium on day 8 post infection.

Still referring to FIG. 1, a plurality of iPSC may be characterized, and a plurality of iPSC may be selected and subjected to 2D expansion. For example, 12 iPSC clones may be selected and subjected to 2D expansion. iPSC colonies may be selected and transferred, for example, on day 21-30 post infection. 2D expansion of a plurality of iPSC may include facilitating the growth of the iPSC along an XY axis. 2D expansion may include transferring iPSC into one or more vessels containing a 2D culture medium. A 2D culture medium may include an Mtesr1 medium. A vessel containing a 2D culture medium may include an extracellular protein matrix (such as laminin, collagen, fibronectin, etc.). A vessel containing a 2D culture medium may include a laminin 521 matrix. 2D expansion may include transferring iPSC into one or more vessels and/or one or more media changes. For example, on day 35 post infection, 6 clones may be transferred into a 6 well plate. For example, on day 40 post infection, 6 clones may be transferred into a 60 mm plate. For example, on day 40-70 post infection, 6 clones may be cycled in a 60 mm plate. For example, from day 75-85 post infection, 3 clones may be expanded from 1 to 2 to 4 to 12 plates. A 2D expansion step may improve confluence, morphology, cleanliness and clear viral vector. A plurality of iPSC may be separated, such as for transfer to a different vessel, using a cutting apparatus.

Still referring to FIG. 1, a plurality of iPSC may be tested post-2D expansion. For example, a plurality of iPSC may undergo one or more of sterility, mycoplasma, karyotype, DNA fingerprinting, residual virus, pluripotency marker, etoposide sensitivity, and thaw grade tests.

Still referring to FIG. 1, iPSC may undergo a sterility test. A sterility test may reveal whether microorganisms are present in an iPSC sample. A sterility test may measure CO2 levels in a sample in order to determine whether microorganisms are present. For example, a CO2 sensor may be used to detect CO2 levels periodically, with an increasing rate of change in CO2 levels indicating the presence of microorganisms. In some embodiments, a sterility test may be done using an automated blood culture system. Automated blood culture may be done using, for example, a VersaTREK automated microbial detection system. In some embodiments, a sterility test may have a negative result. In some embodiments, one or more processes of a sterility test is automated. In some embodiments, incubation and/or CO2 detection is automated, such as via a fluorescent CO2 sensor.

Still referring to FIG. 1, iPSC may undergo a mycoplasma test. A mycoplasma test may reveal whether mycoplasma are present in an iPSC sample. Mycoplasma are very small self-replicating bacteria that may cause changes in cell membranes, nucleic acid and amino acid metabolism, rates of cell growth, and may cause chromosomal defects. Mycoplasma may be detected via direct culture, DNA staining with fluorescent dye, hybridization of nucleic acid, biochemical tests, and PCR. In some embodiments, a mycoplasma test may be done using PCR, for example, using primers associated with a mycoplasma gene. In some embodiments, a mycoplasma test has a negative result. In some embodiments, one or more processes of a mycoplasma test is automated. For example, various aspects of PCR may be automated, such as sample lysis, polynucleotide amplification, and polynucleotide detection.

Still referring to FIG. 1, iPSC may undergo a karyotype test. A karyotype test may measure genomic integrity of an iPSC sample. A karyotype test may be done by G-banding. In some embodiments, G-banding includes staining chromosomes, and visually examining stained metaphase chromosomes for abnormalities such as varying chromosome count or large deletions, insertions, or translocations. In some embodiments, a karyotype test may have a normal result.

Still referring to FIG. 1, iPSC may undergo a DNA fingerprinting test. A DNA fingerprinting test may measure a genotype of an iPSC sample and compare it to a genotype of a parent fibroblast sample. A DNA fingerprinting test may be done using a short tandem repeat (STR) system. In some embodiments, an STR system measures STRs, short DNA sequences repeated in a genome, and determines whether the length of fibroblast STRs match iPSC STRs. In some embodiments, a DNA fingerprinting test may indicate that there is a match with the plurality of fibroblasts the plurality of iPSC was derived from. In some embodiments, PCR may be used to determine STR length. In some embodiments, one or more processes of a DNA fingerprinting test is automated. For example, various aspects of PCR may be automated, such as sample lysis, polynucleotide amplification, and polynucleotide detection.

Still referring to FIG. 1, iPSC may undergo a residual virus test. A residual virus test may measure an amount of viral polynucleotide in an iPSC sample. A residual virus test may measure an amount of viral polynucleotide from a viral vector encoding one or more reprogramming factors. For example, in the case of an RNA viral vector from a Sendai virus, a residual virus test may measure an amount of Sendaiviral RNA in an iPSC sample. In some embodiments, PCR may be used to measure viral DNA (for example, using viral primers). In some embodiments, reverse transcriptase PCR may be used to measure viral RNA (for example, using viral primers). In some embodiments, virus is not detected. In some embodiments, virus is not detected after 40 cycles. In some embodiments, one or more processes of a residual virus test is automated. For example, various aspects of PCR may be automated, such as sample lysis, polynucleotide amplification, and polynucleotide detection.

Still referring to FIG. 1, iPSC may undergo a pluripotency marker test. A pluripotency marker test may measure the amount of cells in a sample that have pluripotent cell (or iPSC) markers. In some embodiments, a pluripotency marker test measures the percent of cells in a sample that express TRA-1-60 and TRA-1-81. In some embodiments, TRA-1-60 and TRA-1-81 are markers for human pluripotent stem cells, such as iPSC. In some embodiments, a pluripotency marker test may be done via flow cytometry (such as using fluorescent antibodies targeting TRA-1-60 and TRA-1-81). In some embodiments, >70% of cells in a sample may express pluripotency markers. In some embodiments, one or more processes of a pluripotency marker test is automated. For example, various aspects of flow cytometry may be automated, such as capturing images of cells, and analyzing those images (such as for fluorescence).

Still referring to FIG. 1, iPSC may undergo an etoposide sensitivity test. An etoposide sensitivity test may measure cell viability using flow cytometry after exposure of cells to etoposide. In some embodiments, iPSC are more susceptible to DNA damaging agents than differentiated cells. In some embodiments, iPSC have lower cell viability after exposure to etoposide. In some embodiments, etoposide is a DNA damaging agent. In some embodiments, an alternative DNA damaging agent, such as topoisomerase II, may be used. In some embodiments, an etoposide sensitivity test may have a EC50<300 nM result. In some embodiments, an etoposide sensitivity test may be used to determine product quality. In some embodiments, one or more processes of a pluripotency marker test is automated. For example, various aspects of flow cytometry may be automated, such as capturing images of cells, and analyzing those images (such as for cell viability).

Still referring to FIG. 1, iPSC may undergo a thaw grade test. A thaw grade test may include a relative determination of iPSC quality based on manual observational assessment. In some embodiments, a thaw grade test may be done by a computer system, such as an artificial intelligence system.

Still referring to FIG. 1, a criteria may include selecting cloned cells based on criteria such as cells that are confluent, have a certain nucleus to cytoplasm ratio, no genomic instability, no DNA damage, “solid edges,” a “homogenous appearance,” and the like. In an embodiment, iPSC may be graded using a criteria, wherein confluent iPSC may be selected as function of the grade. In some instances, this may be done by observational reports done by a human, in other instances this may be done using computer software technology. In some embodiments, a plurality of iPSC may be selected based on a grade. In some embodiments, a plurality of iPSC may be selected based on the results of one or more tests selected from sterility, mucoplasma, karyotype, DNA fingerprinting, residual virus, pluripotency marker, etoposide sensitivity, and thaw grade tests. In some embodiments, a plurality of confluent iPSC may be selected.

Still referring to FIG. 1, a plurality of iPSC may be frozen. For example, a plurality of iPSC may be frozen on day 90 post-infection. A plurality of iPSC may be frozen in a freezing medium, such as Cryotor CS10.

Still referring to FIG. 1, in some embodiments, a method includes a step of differentiating iPSC. In some embodiments, a plurality of iPSC may be differentiated into a cell type selected from the list consisting of cardiac lineage cells, lung cells, liver cells, stomach cells, kidney cells, muscle cells, pancreas cells, skin cells, cartilage cells, and embryonic cells. A plurality of iPSC may be differentiated into cardiac lineage cells.

Differentiating iPSC may include thawing a plurality of iPSC. Differentiating iPSC may include a 2D expansion step. For example, a plurality of iPSC may be thawed and plated on a single plate on day 0, split into 2 plates on day 5, split into 4 plates on day 10, and split into 12 plates on day 15. Plates may include an adhesion protein such as laminin 521. Plates may include a 2D culture medium, which may include Mtesr1. Differentiating iPSC may include a 3D expansion step. For example, a plurality of iPSC may be transferred to a 50 mL vessel on day 20 post thawing, transferred to a 100 mL vessel on day 23 post thawing, transferred to a 200 mL vessel on day 26 post thawing, transferred to a 400 mL vessel on day 29 post thawing, and transferred to a 800 mL vessel on day 32 post thawing. a plurality of iPSC may be in a 3D culture medium during a 3D expansion step. A 3D culture medium may include one or more of Mtesr, DMEM/F12+Glutamax, StemProhESC supplement, BSA 25%, bFGF, and 2-ME.

Still referring to FIG. 1, an iPSC generated by a method described herein may have a variety of applications and therapeutic uses. In some embodiments, a method disclosed herein may be used to reprogram cells suitable for therapeutic applications, including autologous transplantation into subjects.

Still referring to FIG. 1, the methods disclosed herein can be used to generate iPSC that can be further modulated to form any type of somatic cells by culturing the iPSC under cell-type specific conditions. Cell-type or cell lineage specific conditions may include contacting the iPSC with cell and cell lineage differentiation factors. Specifically, iPSC can be differentiated toward a neuronal lineage by exposing them to one or more factors that include, but are not limited to, N2 and B27 supplements, Noggin, SB431542, DMEM/F12 medium, laminin, cyclic adenosine monophosphate (cAMP), ascorbic acid, brain-derived neurotrophic factor (BDNF), glial cell-derived neurotrophic factor (GDNF), insulin-like growth factor I (IGF-I), fibroblast growth factor (FGF)-8, transforming growth factor (TGF) beta 3 (TGF-β3), or retinoic acid. iPSC can be differentiated toward an endodermal lineage (such as hepatocytes, pancreatic cells, intestinal epithelial, lung cells) by exposing them to specific differentiation factors and media, which include, but are not limited to, RPMI medium, SFD medium, N2/B27 medium, glutamine, monothioglycerol (MTG), CHIR 99021, activin A, ascorbic acid, bone morphogenetic protein (BMP)-4, vascular endothelial growth factor (VEGF), epidermal growth factor (EGF), basic FGF (bFGF), hepatocyte growth factor (HGF), dexamethasone, TGF-α, hydrocortisone, FGF-7, or Exendin-4. Cardiomyocyte lineage differentiation factors and media include, but are not limited to, StemPro medium, DMEM/F12 medium, BMP4, Activin A, bFGF, VEGF, Dickkopf-related protein 1 (DKK1), Transferrin, MTG, or ascorbic acid. For mesenchymal stem cell differentiation, iPSC can be exposed to fetal serum and differentiation factors which include, but are not limited to, bFGF, BMP-4, EGF, retinoic acid, or platelet derived growth factor (PDGF). iPSC-derived MSC can subsequently be differentiated toward (1) bone progenitors (osteocytes) through exposure to one or more factors such as ascorbic-acid-2-phosphate, β-glycerophosphate, M dexamethasone or BMP-2, (2) chondrogenic progenitor (chondrocytes) through exposure to one or more factors such as dexamethasone, ascorbic-acid-2-phosphate, proline, pyruvate, TGF-β3, or insulin/transferrin/selenious acid supplement (ITS) (3) adipogenic progenitors through exposure to one or more factors such as hydrocortisone, isobutylmethylxanthine or indomethacin; and (4) fibroblasts through exposure to connective tissue growth factor (CTGF). Fibroblasts can also be derived directly from iPSC via exposure to one or more factors such as TGF-β2, ascorbic acid, connective tissue growth factor (CTGF), ITS reagents, or fetal serum. Keratinocyte lineage differentiation factors include, but are not limited to, BMP4, retinoic acid, ascorbic acid, insulin, hydrocortisone, bovine pituitary extract, IGF-1 or EGF.

Still referring to FIG. 1, a method disclosed herein may also be useful to reprogram or dedifferentiate cells prior to re-differentiation of cells and organ formation. In some embodiments, the methods include generating organs by exposing pluripotent cells generated by the methods described herein to differentiating factors and combining one or more of the differentiated cells and cell types under conditions sufficient to encourage organ formation. For example, iPSC generated using the methods described herein can be differentiated to cells that can be used to make skin as well as other organs such as liver, bones, and cartilage. Such methods include combining one or more of the lineages and/or cell types that form an organ under conditions sufficient to encourage organ formation. Specifically, conditions sufficient to form skin may include but are not limited to co-culture or in vivo co-grafting of iPSC-derived keratinocytes and fibroblasts. For ex vivo generated skin equivalents, fibroblasts are grown on extracellular protein matrix (such as collagen, laminin, fibronectin, etc.) to form a dermis-like structure followed by overlaying with keratinocytes to produce epidermis. For an in vivo generation of human skin equivalents/grafting, a silicone grafting chamber can be surgically inserted onto the muscle fascia of recipient severe combined immunodeficiency (SCID) mice. A cell slurry consisting of keratinocytes and fibroblasts derived from human iPSC is introduced into this chamber. The cells and factors necessary to generate human skin equivalents ex vivo and in vivo include, but are not limited to, iPSC derived keratinocytes, fibroblasts, melanocytes and derma papilla cells, EGF, insulin, fetal serum, ascorbic acid, hydrocortisone, bovine pituitary extract, IGF-1, or DMEM medium. Bones can be grown ex vivo by culturing iPSC-derived osteocytes in the presence of ascorbic-acid-2-phosphate, β-glycerophosphate and fetal serum. Cartilage can be generated by culturing iPSC-derived chondrocytes as micromasses in the presence of ITS, dexamethasone, ascorbic-acid-2-phosphate, proline, pyruvate and TGF-β3. Liver can be generated via the formation of liver buds. Conditions sufficient to form liver buds may include, but are not limited to, the combination of mesenchymal stem cells with hepatic progenitors (both can be derived from iPSC as described above) in the presence of endothelial growth medium and/or hepatocyte culture medium supplemented with dexamethasone, oncostatin, HGF, and matrigel.

Still referring to FIG. 1, in some embodiments, a method for treating or preventing one or more symptoms of a disease or disorder in a subject may include reprogramming cells to pluripotency in vitro, differentiating the cells to one or more appropriate cell types, and administering a therapeutically effective amount of differentiated cells to a subject in need thereof. A method may include obtaining one or more somatic cells from a subject and reprogramming the cells into iPSC or dedifferentiated cells. Cells may be cultured under conditions that allow for the cells to differentiate into a desired cell type suitable for treating or preventing a condition. Differentiated cells may be introduced into a subject to treat or prevent a condition. For example, a method may include obtaining one or more somatic cells from a subject, reprogramming the somatic cells into iPSC, differentiating the iPSC into a desired cell type, and administering a therapeutically effective amount of differentiated cells to the subject.

Still referring to FIG. 1, in some embodiments, the methods disclosed herein may yield reprogrammed cells with normal karyotypes or with karyotypes that are the same as the subject from whom they were derived. In some embodiments, uncorrected iPSC from subjects may be differentiated into cell types relevant to a genetic disorder for modeling a disease. In another embodiment, a particular mutation of interest may be introduced into normal healthy iPSC, as another approach to modeling a disorder.

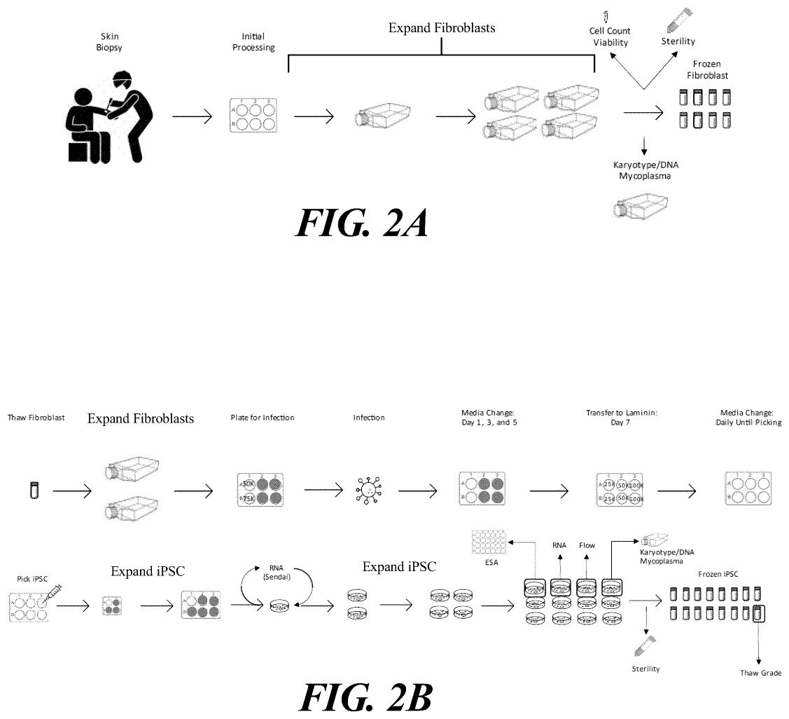

Now referring to FIG. 2A, in some embodiments, a method includes, in the following order, receiving fibroblasts from a skin biopsy, an initial processing step, an expansion step, and a testing step. In some embodiments, fibroblasts are then frozen.

Now referring to FIG. 2B, in some embodiments, a method includes, in the following order, thawing fibroblasts, expanding fibroblasts, plating fibroblasts for infection, infecting fibroblasts, media changes, transfer to a laminin plate, media changes, a picking step, expansion, a testing step, and freezing iPSC.

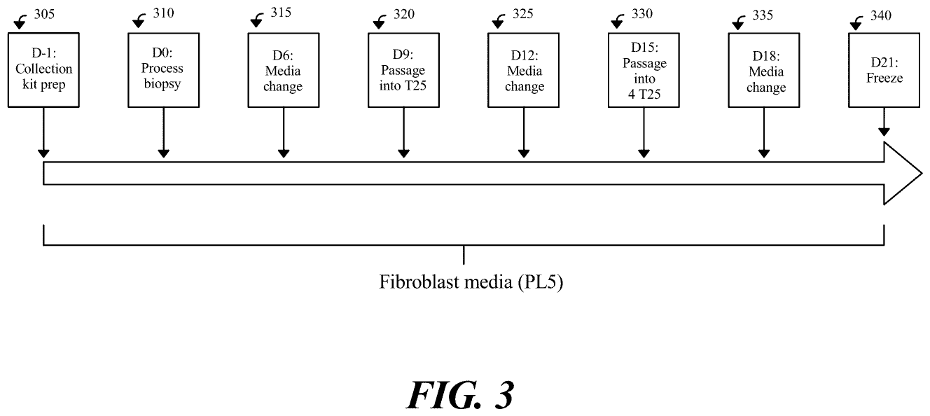

Referring now to FIG. 3, an exemplary embodiment of a method of collecting, expanding, and freezing fibroblast cells is disclosed. Fibroblast cells may be collected via skin biopsy. A collection kit may be prepared on day-1 (305). A biopsy may be processed on day 0 (310). Media may be changed on day 6 (315). Cells may be transferred into a T25 flask on day 9 (320). Media may be changed on day 12 (325). Cells may be split and transferred into 4 T25 flasks on day 15 (330). Media may be changed on day 18 (335). Cells may be frozen on day 21 (340).

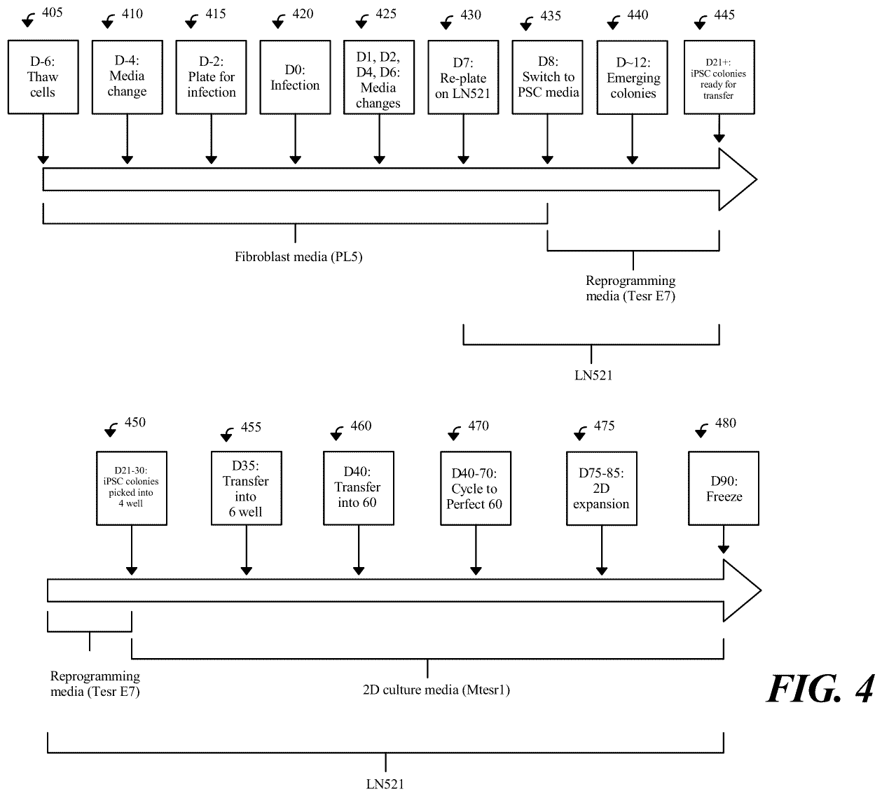

Referring now to FIG. 4, a method of reprogramming cells is disclosed. In some embodiments, a method may start with a sample of frozen fibroblasts. Fibroblasts may be thawed on day-6 (405). Media may be changed on day-4 (410). Cells may be plated for infection on day-2 (415). Cells may be infected (such as with a viral vector encoding one or more reprogramming factors) on day 0 (420). Media may be changed on days 1, 2, 4, and 6 (425). Cells may be re-plated on a matrix such as LN521 (430). Cells may be switched to PSC media such as Tesr E7 on day 8 (435). iPSC colonies may emerge or may be detected on about day 12 (440). iPSC colonies may be ready for transfer on at least day 21 (445). iPSC colonies may be picked into 4 wells on day 21-30 (450). Cells may be transferred into 6 wells on day 35 (455). Cells may be transferred into a 60 mm plate on day 40 (460). Cells may be cycled to perfect 60 from day 40 to 70 (470). Cells may be expanded via 2D expansion from day 75-85 (475). Cells may be frozen on day 90 (480).

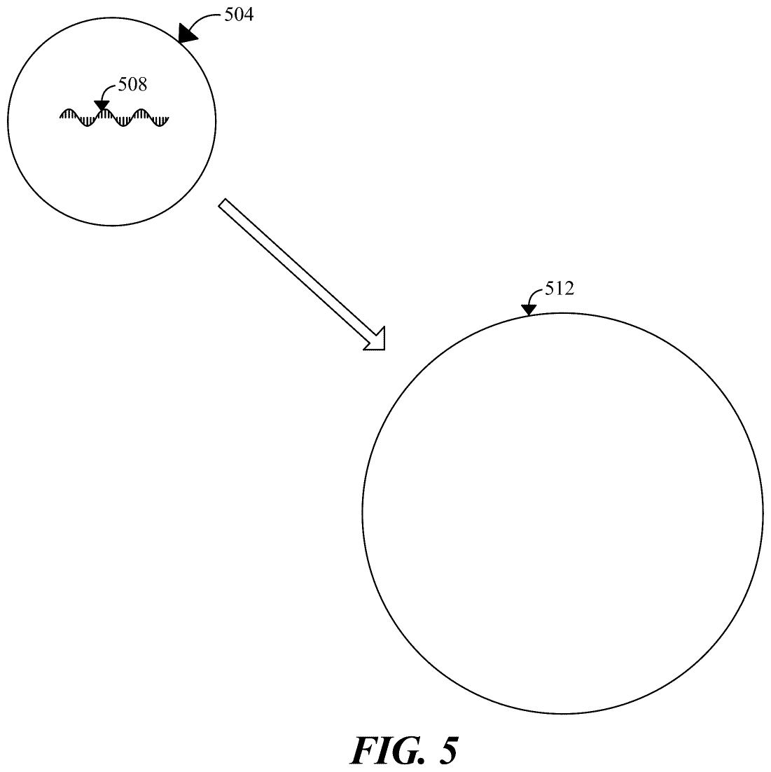

Referring now to FIG. 5, a polynucleotide 508, such as a polynucleotide encoding one or more reprogramming factors, may be delivered to a cell 512, such as a fibroblast, via a viral vector. A polynucleotide may be RNA, such as mRNA. A viral vector may include a viral particle 504.

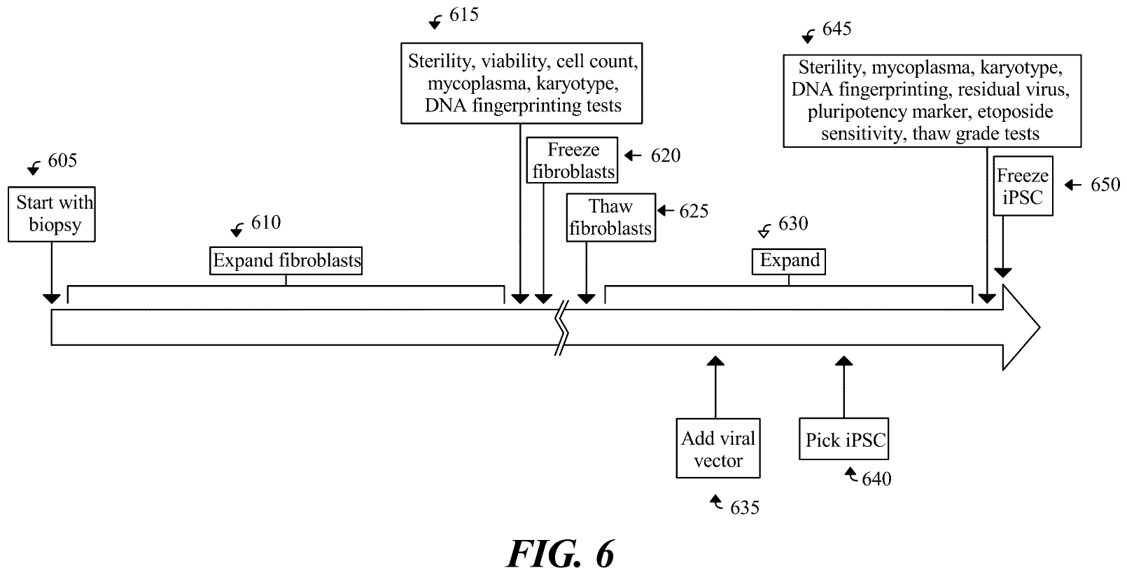

Referring now to FIG. 6, a method of reprogramming cells is disclosed. Starting material may include a skin biopsy (605). Fibroblasts may be expanded (610). Sterility, viability, cell count, mycoplasma, karyotype, and DNA fingerprinting tests may be done (615). Fibroblasts may be frozen (620). Fibroblasts may be thawed (625). Cells may be expanded (630). A viral vector may be added (635). iPSC may be picked (640). Sterility, mycoplasma, karyotype, DNA fingerprinting, residual virus, pluripotency marker, etoposide sensitivity, and thaw grade tests may be done (645). iPSC may be frozen (650).

As used herein, the terms “administer,” “administering,” “administration,” or the like refer to the placement of a composition into a subject by any method. A composition described herein may be administered to a subject by any one of a variety of manners or a combination of varieties of manners. For example, a composition may be administered orally, nasally, intraperitoneally, or parenterally, by intravenous, intramuscular, topical, or subcutaneous routes, or by injection into tissue.

As used herein, “effective amount” or “therapeutically effective amount” is the amount of a composition of this disclosure which, when administered to a subject, is sufficient to effect treatment of a disease or condition in the subject. The amount of a composition of this disclosure which constitutes a “therapeutically effective amount” will vary depending on the composition, the condition and its severity, the manner of administration, and the age of the subject to be treated.

As used herein, “treating” or “treatment” means the treatment of a disease or condition of interest in a subject having the disease or condition of interest, and includes: (i) preventing the disease or condition from occurring in the subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, i.e., relieving pain without addressing the underlying disease or condition.

Referring back now to FIG. 1, an exemplary embodiment of a method of reprogramming cells is illustrated. In some embodiments, a method utilizes a computing device. A computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. A computing device may include a single computing device operating independently, or may include two or more computing device operating in concert, in parallel, sequentially or the like; two or more computing devices may be included together in a single computing device or in two or more computing devices. A computing device may interface or communicate with one or more additional devices as described below in further detail via a network interface device. Network interface device may be utilized for connecting a computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. A computing device may include but is not limited to, for example, a computing device or cluster of computing devices in a first location and a second computing device or cluster of computing devices in a second location. A computing device may include one or more computing devices dedicated to data storage, security, distribution of traffic for load balancing, and the like. A computing device may distribute one or more computing tasks as described below across a plurality of computing devices of computing device, which may operate in parallel, in series, redundantly, or in any other manner used for distribution of tasks or memory between computing devices. A computing device may be implemented using a “shared nothing” architecture in which data is cached at the worker, in an embodiment, this may enable scalability of method 100 and/or computing device.

With continued reference to FIG. 1, a computing device may be designed and/or configured to perform any method, method step, or sequence of method steps in any embodiment described in this disclosure, in any order and with any degree of repetition. For instance, a computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

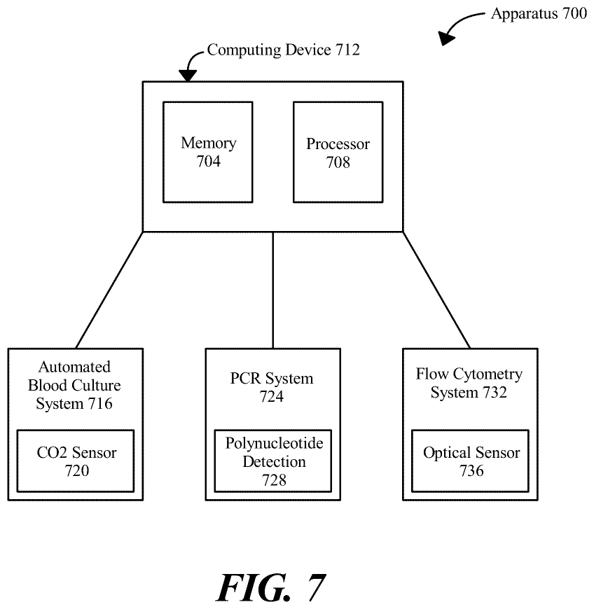

Now referring to FIG. 7, in some embodiments, an apparatus 700 includes a computing device 712, an automated blood culture system 716, a PCR system 724, and a flow cytometry system 732. In some embodiments, a computing device 712 includes a memory 704 and a processor 708. In some embodiments, an automated blood culture system 716 includes a CO2 sensor 720. In some embodiments, a PCR system 724 includes a polynucleotide detection system 728. In some embodiments, a flow cytometry system 732 includes an optical sensor 736. In some embodiments, a feature depicted in FIG. 7 may be implemented as described with reference to FIG. 1.

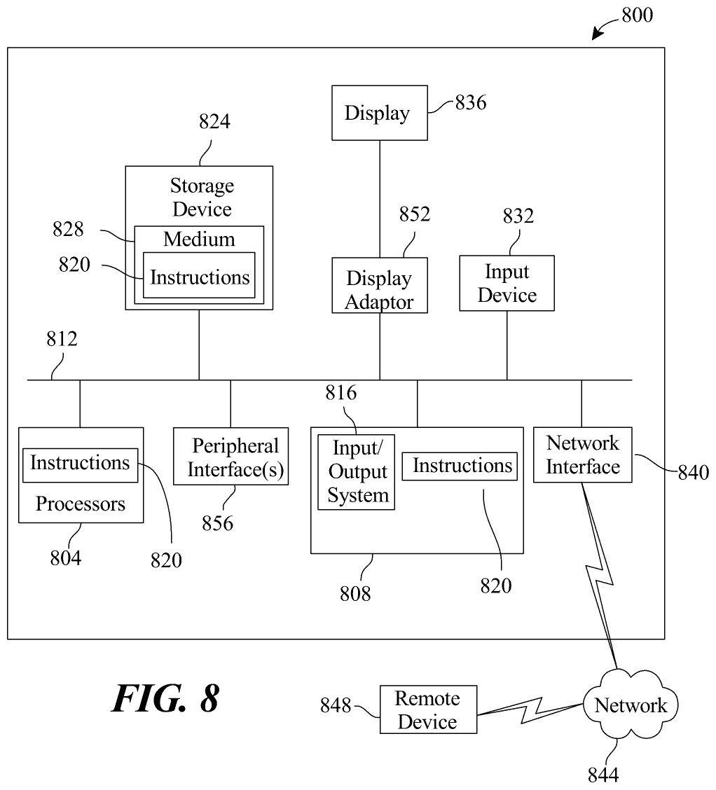

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve compositions and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method of reprogramming cells, the method comprising:
obtaining a plurality of fibroblast cells;
expanding the plurality of fibroblast cells, wherein expanding the plurality of fibroblast cells comprises a first expansion step comprising at least one medium change for a first growth medium for the first expansion step;
reprogramming the plurality of fibroblast cells, wherein reprogramming the plurality of fibroblast cells comprises:
delivering, to the plurality of fibroblast cells, a polynucleotide encoding a reprogramming factor;
transferring the plurality of fibroblast cells to a reprogramming medium comprising a vitamin C derivative; and
generating a plurality of induced pluripotent stem cells (iPSC) as a function of transferring the plurality of fibroblast cells to the reprogramming medium;
expanding the plurality of iPSC; and
identifying a plurality of confluent iPSC from the plurality of iPSC.

2. The method of claim 1, wherein the polynucleotide encoding a reprogramming factor is RNA.

3. The method of claim 1, wherein obtaining the plurality of fibroblast cells further comprises obtaining the plurality of fibroblast cells using a skin biopsy.

4. (canceled)

5. The method of claim 1, wherein expanding the plurality of fibroblast cells comprises transferring the plurality of fibroblast cells to a vessel comprising an extracellular protein matrix.

6. The method of claim 1, wherein the reprogramming factor is selected from the list consisting of h(human gene)Oct3/4, h(human gene)Sox2, h(human gene)Klf4, and h(human gene)c-Myc.

7. The method of claim 6, wherein the polynucleotide encoding a reprogramming factor encodes each of h(human gene)Oct3/4, h(human gene)Sox2, h(human gene)Klf4, and h(human gene)c-Myc.

8. The method of claim 1, wherein the polynucleotide encoding a reprogramming factor is delivered to the plurality of fibroblast cells via a viral vector.

9. The method of claim 8, wherein the polynucleotide encoding a reprogramming factor is delivered to the plurality of fibroblast cells via a Sendai viral vector.

10. The method of claim 1, wherein identifying a plurality of confluent iPSC from the plurality of iPSC comprises subjecting the iPSC to a quality test; and selecting the iPSC as a function of the quality test.

11. The method of claim 1, wherein identifying a plurality of confluent iPSC from the plurality of iPSC comprises subjecting iPSC to a test selected from the list consisting of sterility, mycoplasma, karyotype, DNA fingerprinting, residual virus, pluripotency marker, etoposide sensitivity, and thaw grade tests, and selecting iPSC.

12. The method of claim 1, wherein identifying a plurality of confluent iPSC from the plurality of iPSC comprises subjecting iPSC to a pluripotency marker test and selecting iPSC.

13. The method of claim 1, wherein identifying a plurality of confluent iPSC from the plurality of iPSC comprises subjecting iPSC to an etoposide sensitivity test and selecting iPSC.

14. The method of claim 1, wherein identifying a plurality of confluent iPSC from the plurality of iPSC comprises subjecting iPSC to sterility, mycoplasma, karyotype, DNA fingerprinting, residual virus, pluripotency marker, etoposide sensitivity, and thaw grade tests, and selecting iPSC.

15. The method of claim 1, further comprising freezing the plurality of confluent iPSC.

16. The method of claim 1, further comprising differentiating the plurality of confluent iPSC into a plurality of cells having a somatic cell type.

17. The method of claim 16, further comprising administering a therapeutically effective amount of the plurality of differentiated cells to a subject.

18. The method of claim 17, wherein the differentiated cells are autologous with respect to the subject.

19. The method of claim 1, further comprising differentiating the plurality of confluent iPSC into a cardiac lineage cell.

20. The method of claim 19, further comprising administering a therapeutically effective amount of the plurality of differentiated cells to a subject.

21. The method of claim 20, wherein the differentiated cells are autologous with respect to the subject.
